(19) United States
(12) Patent Application Publication
(10) Pub. No.: US 2022/0162689 A1
(43) Pub. Date: May 26, 2022

(54) METHOD AND DEVICE FOR SEQUENCING AND DETECTING DNA/RNA DIRECTLY ON A SENSOR

(71) Applicant: QIAGEN Sciences, LLC, Germantown, MD (US)

(72) Inventors: Michel Perbost, Woburn, MA (US); Jerzy Olejnik, Brookline, MA (US)

(21) Appl. No.: 17/101,958

(22) Filed: Nov. 23, 2020

(51) Int. Cl. C12Q 1/6869

(52) U.S. Cl. CPC C12Q 1/6869; C12Q 2565/607

(57) ABSTRACT

The present invention is related to DNA/RNA sequencing and in particular approaches termed Next Generation Sequencing (NGS). Improved methods and integrated devices are disclosed, which enable faster and more efficient analysis of nucleic acids. One aspect of the invention has to do with integration of detection system into consumable flowcell. This enables miniaturization and simplification of sequencing instrumentation while simultaneously reducing sequencing costs. By performing sequencing and detection directly on the sensor, such as CCD or CMOS, one can significantly speed up the sequencing process.

### BACKGROUND OF INVENTION

### Field of Invention

The present invention relates to DNA/RNA sequencing and in particular approaches termed Next Generation Sequencing (NGS).

### Brief Description of Related Art

Nucleotides are introduced together but different colors differentiate each other. Or the nucleotides are added sequentially on the surface one after each other. Their color is either different or disappear before the next nucleotide is added. The surface is washed in between each addition of 4 different colors, one for each nucleotides, mixed together.

### BRIEF SUMMARY OF THE INVENTION

The present invention is related to DNA/RNA sequencing and in particular approaches termed Next Generation Sequencing (NGS). Improved methods and integrated devices are disclosed, which enable faster and more efficient analysis of nucleic acids.

One aspect of the invention has to do with integration of detection system into consumable flowcell. This enables miniaturization and simplification of sequencing instrumentation while simultaneously reducing sequencing costs. By performing sequencing and detection directly on the sensor, such as CCD or CMOS, one can significantly speed up the sequencing process.

One example of such process involves generation of clonally amplified DNA directly on CMOS sensors or populating each pixel with amplified DNA. Approaches to clonal amplification are well known in the art and can include PCR, RCA and RPA approaches as examples. CMOS sensors require chemical modifications to attach DNA or perform amplification. These chemical modifications include for example, poly-amine modifications for electrostatic attachment of DNA or other chemical modifications, which result in chemical crosslinking (amine to carboxyl or azide to alkyne).

The sequencing reaction may utilize a variety of approaches, for example sequencing by synthesis (SBS), sequencing by ligation (SBL), sequencing by binding (SBB) or sequencing by hybridization (SBH). One preferred embodiment of this invention is SBS. One particularly preferred embodiment is SBS with reversibly terminating nucleotides due to its robustness and ability to resolve homopolymer regions. Reversibly terminating groups are well known in the art and include: aminoxy, azidomethyl, dithio based groups and other groups that can be incorporated into DNA by polymerase and removed in a fashion that does not damage DNA.

In a typical SBS with reversibly terminating nucleotides one can use fluorescence for detection. This allows for 4-color fluorescence based systems to decode 4 different nucleotides in the DNA. To speed up the detection process, approaches using combination of florescence and dark state detection have been described.

In the present embodiment a variety of detection modes are possible. One of the detection modes is fluorescence where a signal can be generate by labels incorporated into DNA during SBS such as via nucleotides. Other potential modes of detection include luminescence or chemiluminescence. For these detection modes additional enzymes are required. These enzymes can be directly or indirectly attached to the nucleotides (e.g., via antibodies, haptens or affinity tags).

The foregoing and other features of the invention are hereinafter more fully described below, the following description setting forth in detail certain illustrative embodiments of the invention, these being indicative, however, of but a few of the various ways in which the principles of the present invention may be employed.

### BRIEF DESCRIPTION OF THE DRAWINGS

FIG. 1 schematically illustrates SBS on CMOS, where a DNA template is attached to each sensor/pixel.

FIG. 2 schematically illustrates an embodiment of the invention in which detection is performed following nucleotides carrying the same label sequentially.

FIG. 3 schematically illustrates an embodiment of the invention in which four separate solutions containing each nucleotide and polymerase are sequentially flowed into a flowcell comprising CMOS sensor.

FIG. 4 schematically illustrates a second embodiment in which nucleotides are ranked from slow to incorporate to fast to incorporate.

### DETAILED DESCRIPTION OF THE INVENTION

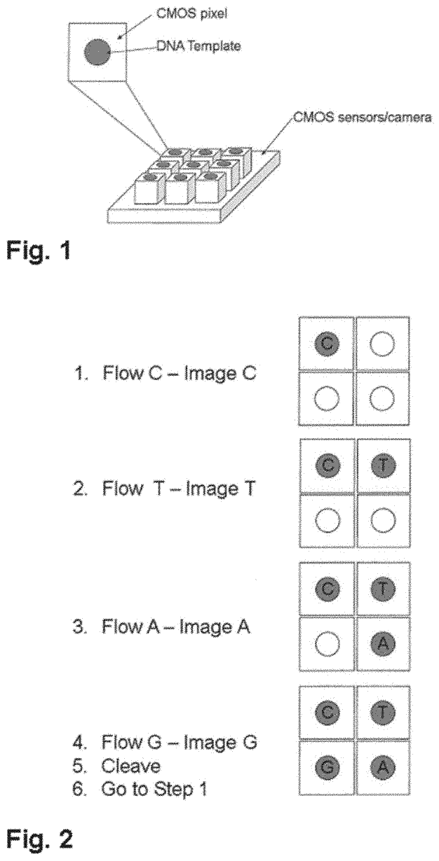

FIG. 1 schematically illustrates SBS on CMOS, where a DNA template is attached to each sensor/pixel. In one embodiment of the invention, detection is performed by flowing nucleotides carrying the same label sequentially. This embodiment is schematically illustrated in FIG. 2.

In another embodiment of the invention, the label is introduced in the separate steps, e.g., an affinity tag carrying the label and capable of recognizing specific bases.

Single color or limited color sequencing by successive introduction of NTP inside a chamber. These NTP can, but need not be, fluorescent. But can be detected. In another embodiment the CMOS constantly monitors the signal during incorporation. For example:
Nucleotides incorporation in sequencing DNA is constantly monitored by a static imaging system located below the DNA
Each nucleotide is introduced in the sequencing chamber, one at a time. The signal is constantly monitored, and when the signal of multiple pixels are saturated for one nucleotide, the solution is replaced with the next nucleotides which will saturate other locations, and so on. When all 4 have been introduced and each DNA location identified for having incorporated 1 of the 4 nucleotides, the dye (if it is a dye), and the 3′blocking group, are cleaved. The cleave step is stopped when fluorescence or chemiluminescence or other detectable property, is back to the baseline. Then the cycle is repeated.

In another embodiment the system leverages different kinetics of incorporation by different nucleotides:
Nucleotides incorporation in sequencing DNA is constantly monitored by a static imaging system located below the DNA
Nucleotides are ranked in order of speed of incorporation. For instance one can have A<T<G<C ranking
   The slowest nucleotide will be introduced first to give it enough time to incorporate and when the signal is strong enough to detect, then, the second nucleotide is introduced − in the example above the slower would be A nucleotide
   A+T mixture is introduced then. The slower + the nucleotide just above in speed of incorporation (T) are introduced then. In the example above that would be A+T
   Then in the next step a mixture of A+T+G is introduced
   Lastly all 4 nucleotide mixture is introduced and shortly after detection of fastest incorporating nucleotide is complete.
The signal is constantly detected, and when the signal of multiple pixels are strong enough for first solution, the solution is replaced with the next solution which has 2 nucleotides which will keep saturating the first location, but will start to incorporate in other. Then 3 are added and finally all 4 nucleotides are added. Then the signal generating system and the 3′blocking group, are cleaved. The cleave step is stopped when fluorescence/signal, or method of detection is back to the baseline. Then the cycle of sequencing is repeated

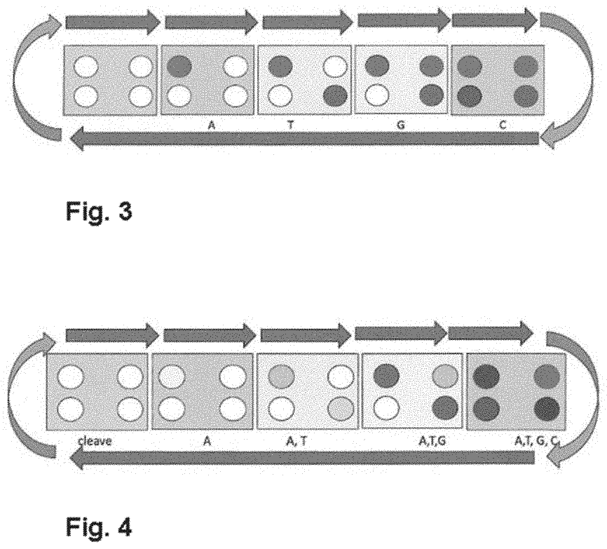

FIG. 3 schematically illustrates an embodiment in which SBS is performed on a CMOS with reversibly terminating nucleotides (with 3′OH blocking groups):
1. 4 separate solutions containing each nucleotide and polymerase sequentially flowed into flowcell comprising CMOS sensor. Signal capture is performed after each flow. The identity of an incorporated nucleotide is deduced based on appearance of the signal after the flow.
   a. A, T, G and C can be used with same label/signal (shown in FIG. 3 with different shading to make it easier to illustrate the process).
2. Rolonies/clonally amplified DNA on the surface in white holes representing a CMOS pixel.
3. Solution A is first introduced in flow cell/and signal collected.
4. Solution Tis then introduced.
5. Solution G is then introduced.
6. Solution C is then introduced.
7. Then dye and 3′0H are cleaved and cycle restart.

FIG. 4 schematically illustrates a second embodiment of the invention.
1. Nucleotides are ranked from slow to incorporate to fast to incorporate.
2. If A is slower than T, which is slower than G which is slower than C, 4 solutions needs to be prepared:
   a. 1 A
   b. 2 A+T
   c. 3 A+T+G
   d. 4 A+T+G+C.
3. They are introduced one after the other as soon the signal can be detected in the rolonies and before addition is completed. Later solutions will complete the signal.
4. Rhythm of solution introduction can change as function of what is detected on CMOS.
5. The second embodiment is likely faster than the first, but still requires completion of incorporation even after detection is accomplished to prevent any phasing issues.

In both cases the amount of time to incorporate can be adjusted by constantly monitoring the fluorescence on the surface.

Additional advantages and modifications will readily occur to those skilled in the art. Therefore, the invention in its broader aspects is not limited to the specific details and illustrative examples shown and described herein. Accordingly, various modifications may be made without departing from the spirit or scope of the general inventive concept as defined by the appended claims and their equivalents.

What is claimed is:

1. Methods and devices for carrying out the invention in the broadest scope permissible under U.S. law.
